(19) United States
(12) Patent Application Publication
(10) Pub. No.: US 2015/0045564 A1
(43) Pub. Date: Feb. 12, 2015

(54) Cu-MEDIATED ANNULATION FOR THE EFFECTIVE SYNTHESIS OF 3-SUBSTITUTED PHTHALIDES

(71) Applicant: Council of Scientific & Industrial Research, New Delhi (IN)

(72) Inventors: Santhosh Rekula Reddy, Pune (IN); Kishore Prasad Pragati, Pune (IN); Kiran Naga, Pune (IN); Arumugam Sudalai, Pune (IN)

(21) Appl. No.: 14/370,441

(22) PCT Filed: Jan. 3, 2013
(86) PCT No.: PCT/IN2013/000005

§ 371 Date: Jul. 2, 2014

(30) Foreign Application Priority Data
Jan. 3, 2012 (IN) 0011/DEL/2012

(51) Int. Cl. C07D 307/88; C07D 307/89

(52) U.S. Cl. CPC C07D 307/88; C07D 307/89
USPC 549/310; 549/307

(57) ABSTRACT

The present invention disclosed herein is a novel commercially feasible, one pot synthesis of library of 3-substituted phthalides of formula I via CuCN mediated oxidative cyclization in high yield. Formula I

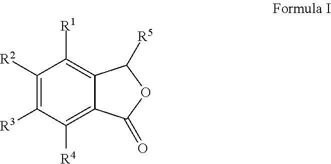

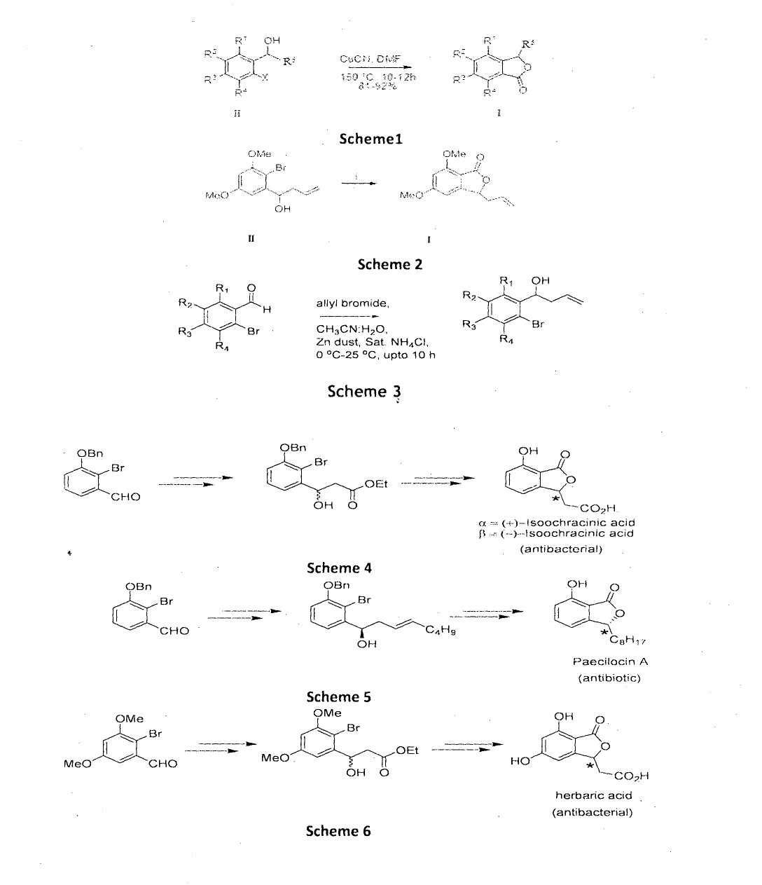

The following specification particularly describes the invention and the manner in which it is to be performed:

### FIELD OF THE INVENTION

The present invention relates to commercially feasible one pot synthesis of library of 3-substituted phthalides of formula I via CuCN mediated oxidative cyclization in high yield.

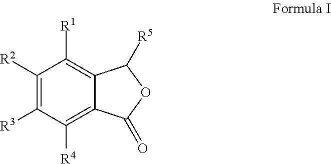

### BACKGROUND AND PRIOR ART OF THE INVENTION

A wide range of natural products with broad, potent, and potentially path-pointing biological activities possess 3-substituted phthalide core. For example, the natural products like isochracinic acid, paecilocin A and herbaric acid which possess 3-substituted phthalide core have antibacterial, antifungal, antibiotic activity. The development of efficient synthetic procedures for the facile construction of this important molecular framework is an important goal in organic synthesis. Inventions have been made in the synthesis of phthalides that exhibited antibacterial and antifungal activity on antimicrobial screening against human pathogenic bacteria and fungi.

An article titled “Rhodium or palladium-catalyzed cascade aryl addition/intramolecular lactonization of phthalaldehydronitrile to access 3-aryl and 3-alkenyl phthalides” by Guanglei Lva, Genping Huanga et. al in Tetrahedron Volume 67, Issue 26, 1 Jul. 2011, Pages 4879-4886 discloses a rhodium or palladium-catalyzed addition of boronic acids to phthalaldehydronitrile, followed by an intramolecular lactonization of cyano to access 3-substituted phthalides.

An article titled “Synthesis of Chiral 3-Substituted Phthalides by a Sequential Organocatalytic Enantioselective Aldol-Lactonization Reaction. Three-Step Synthesis of (S)-(−)-3-Butylphthalide” by Haoyi Zhang, Shilei Zhang et. al in J. Org. Chem., 2010, 75 (2), pp 368-374 discloses organocatalytic asymmetric aldol-lactonization reaction of 2-formylbenzoic esters with ketones/aldehydes for construction of the enantioenriched 3-Substituted Phthalides. The said article further describes 3-step catalytic asymmetric synthesis of the natural product of 3-butylphthalide.

Article titled “Phthalides by Rhodium-Catalyzed Ketone Hydroacylation” by Diem H. T. Phan J. Am. Chem. Soc., 2009, 131 (43), pp 15608-15609 discloses enantioselective intramolecular ketone hydroacylation of various 2-ketobenzaldehydes in the presence of Rh[(Duanphos)]X (X=NO3, OTf, OMs) to produce phthalide.

“An Efficient Synthesis Of 3-Substituted 3H-Isobenzofuran-1-Ylidenamines By The Reaction Of 2-Cyanobenzaldehydes With Organolithiums And Their Conversion Into Isobenzofuran-1(3h)-Ones” by Kazuhiro Kobayashi in Heterocycles, Vol. 83, No. 1, 2011 discloses synthesis of 3-substituted 3H-isobenzofuran-1-ylidenamines by the reaction of 2-cyanobenzaldehydes with nucleophiles, such as organolithiums or lithium enolates of t-butyl acetate and N,N dimethylacetamide and further conversion to the corresponding 3-substituted isobenzofuran-1(3H)-ones (phthalides) upon treatment with hydrochloric acid.

The reported methods for the synthesis of 3-substituted phthalides employ either lithiation followed by carboxylation or carbamate formation followed by lithiation. This limits the broad substrate scope and higher reaction stereoselectivity. The great challenges are remaining since the cyano group is inert to the insertion of metal species in comparison with C═O, partly due to its low polarity. Moreover, the aromatic nitriles may also have good affinity to transition-metals, resulting in the deactivation of the catalyst. In this context, a more practical and efficient synthesis of functionalized 3-substituted phthalide derivatives is highly desirable using less number of reagents via CN activation.

The processes described above are further lengthy, require chiral auxiliaries or chiral organometallics and few are catalytic thus making them costly and industrially non feasible.

The present inventors have now developed a cheaper and practical protocol for the construction of a wide variety of 3-substituted phthalides and their structural analogues, which have promising pharmacological utility that proceeds with high yields in a single step.

### OBJECTS OF THE INVENTION

Main objective of the present invention is to provide one pot synthesis of library of 3-substituted phthalides of formula I via CuCN mediated oxidative cyclization.

### BRIEF DESCRIPTION OF THE DRAWINGS

Scheme 1 represents step for the preparation of compound of formula I.

Scheme 2 represents synthesis of allyl alcohols.

Scheme 3 represents step for the preparation of 3-substituted phthalides; wherein (i) CuCN (3.0 equiv.), DMF, 150° C., 10 h, 86%.

Scheme 4 represents first Asymmetric synthesis of (+) & (−)-Isochracinic acid.

Scheme 5 represents first Asymmetric Synthesis of Paecilocin A.

Scheme 6 represents First Asymmetric Synthesis of Herbaric acid.

### SUMMARY OF THE INVENTION

Accordingly, present invention provides a one pot process for preparation of 3-substituted phthalides of formula I and their structural analogues and the said process comprising the steps of reacting halo alcohols of a compound of formula II with CuCN in the ratio ranging between 3.0 to 3.1 in polar aprotic solvent at a temperature in the range of 145° C.-155° C. for a time period in the range of 10-13 hours;

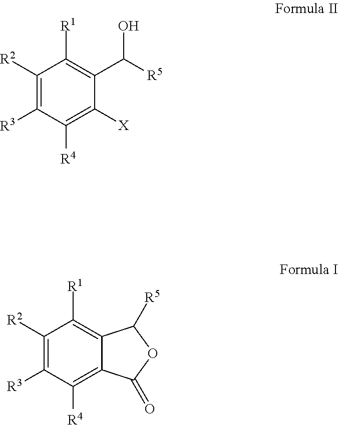

wherein R¹, R², R³, R⁴ are selected independently from hydrogen, halogen, hydroxy, cyano, carboxyl, amino or substituted amino, alkyl, alkenyl, alkynyl, alkoxy, alkenyloxy, alkynyloxy, alkylthio, alkenylthio, alkynylthio, aryl, aralkyl, aralkenyl, aralkynyl, aryloxy, aralkoxy, acyl, acylalkyl, acyloxy, acyloxyalkyl, hydroxyalkyl, cycloalkyl, alkylthio, alkylsulfonyl, alkylsulfinyl, which may optionally be substituted with halo, hydroxy, alkyl, alkoxy, nitro, amino, cyano, carboxy;

R5 is selected independently from alkyl, allyl, vinyl, alkynyl, aryl, alkenyl, which may optionally be substituted with halo, hydroxy, alkyl, alkoxy, nitro, amino, cyano, carboxy; acyloxyalkyl, acyloxyaryl,

X represents a halo group;

with the proviso, that when R² and R³ together represent —O—CH₂—O—, R¹ and R⁴ are hydrogen, R⁵ is selected independently from alkyl, allyl, vinyl, alkynyl, aryl, alkenyl, which may optionally be substituted with halo, hydroxy, alkyl, alkoxy, nitro, amino, cyano, carboxy; acyloxyalkyl or acyloxyaryl;

with the proviso that when R¹, R², R³ and R⁴ together represent the group selected from (E)-ethyl 3-(1-cyanonaphthalen-2-yl)acrylate; 3-(1-hydroxybut-3-enyl)pyridine-2-carbonitrile; 1-(3-bromofuran-2-yl)but-3-en-1-ol; R⁵ is selected independently from alkyl, allyl, vinyl, alkynyl, aryl, alkenyl, which may optionally be substituted with halo, hydroxy, alkyl, alkoxy, nitro, amino, cyano, carboxy; acyloxyalkyl or acyloxyaryl

with the proviso when R¹ to R⁴ are similar or independently selected from the group consisting of hydrogen, then R⁵ is particularly selected from the group consisting of (C¹-C¹⁶) alkyl, (C²-C⁸) alkynyl, carboxylate, vinyl, aryl, alkylaryl, optionally be substituted with hydrogen, halo, hydroxy, alkyl, alkoxy, nitro, amino, cyano and like thereof.

In an embodiment of the present invention, polar aprotic solvent used is selected from the group consisting of Dimethylformamide (DMF), Dichloromethane (DCM), acetone, tetrahydrofuran (THF) or acetonitrile.

In yet another embodiment of the present invention, the halo group is selected from chloro, bromo or iodo preferably bromo.

In yet another embodiment of the present invention, the compound of formula II optionally is prepared by subjecting o-bromoaldehydes to Barbier allylation or Grignard reaction using allylbromide or alkyl halides.

In yet another embodiment of the present invention, 3-substituted phthalides of formula I comprising:
i. 3-Allylisobenzofuran-1-one;
ii. 3-Allyl-5-methoxyisobenzofuran-1-one;
iii. 3-Allyl-5,7-dimethoxyisobenzofuran-1-one;
iv. 3-Allyl-4,5,6-trimethoxyisobenzofuran-1-one;
v. 1-Allyl-1,3-dihydro-5-methoxy-3-oxoisobenzofuran-6-yl4-methylbenzenesulfonate;
vi. 3-Allyl-6-methoxy-5-phenoxyisobenzofuran-1-one;
vii. 3-Allyl-5-fluoroisobenzofuran-1(3H)-one;
viii. 7-Allylisobenzofuro[5,6-d][1,3]dioxol-5(7H)-one;
ix. 3-Methyl-3H-isobenzofuran-1-one; 3-Ethyl-3H-isobenzofuran-1-one;
x. 3-Propyl-3H-isobenzofuran-1-one; 3-Butylisobenzofuran-1(3H)-one;
xi. 3-Heptylisobenzofuran-1(3H)-one.

In yet another embodiment of the present invention, yield of compound of formula is in the range of 80 to 92%.

In yet another embodiment of the present invention, the compound of formula II is selected from the group consisting of 1-(2-Bromophenyl)but-3-en-1-ol; 1-(2-Bromo-5-methoxyphenyl)but-3-en-1-ol; 1-(2-Bromo-3,5-dimethoxyphenyl)but-3-en-1-ol; 1-(2-Bromo-3,4,5-trimethoxyphenyl)but-3-en-1-ol; 4-Bromo-5-(1-hydroxybut-3-enyl)-2-methoxyphenyl-4-methylbenzenesulfonate; 1-(2-Bromo-4-methoxy-5-phenoxyphenyl)but-3-en-1-ol; 1-(2-Bromo-5-fluorophenyl)but-3-en-1-ol; 1-(6-Bromobenzo[d][1,3]dioxol-5-yl)but-3-en-1-ol; 1-(2-Bromophenyl)ethanol; 1-(2-Bromophenyl)-1-propanol; 1-(2-Bromophenyl)butan-1-ol; 1-(2-Bromophenyl)pentan-1-ol; 1-(2-Bromophenyl)octan-1-ol etc.

In yet another embodiment of the present invention, the preparation of 3-substituted phthalides of formula I comprising subjecting the bromo alcohols of formula II to Rosenmund-von Braun reaction in the presence of CuCN in DMF under reflux condition.

In yet another embodiment, present invention provides a compound of formula I comprises
a) 3-Allyl-5-methoxyisobenzofuran-1-one;
b) 3-Allyl-4,5,6-trimethoxyisobenzofuran-1-one;
c) 1-Allyl-1,3-dihydro-5-methoxy-3-oxoisobenzofuran-6-yl4-methylbenzenesulfonate;
d) 3-Allyl-6-methoxy-5-phenoxyisobenzofuran-1-one;
c) 3-Allyl-5-fluoroisobenzofuran-1(3H)-one;
f) 7-Allyl isobenzofuro[5,6-d][1,3]dioxol-5(7H)-one;
g) 3-Ethyl-3H-isobenzofuran-1-one;
h) 3-Propyl-3H-isobenzofuran-1-one.

### DETAILED DESCRIPTION OF THE INVENTION

The present invention preferably provides cheap, easy to perform at higher scales, a one pot CuCN-mediated annulation of compound of formula II for the synthesis of a library of 3-substituted phthalides of Formula I and their structural analogues in high yield.

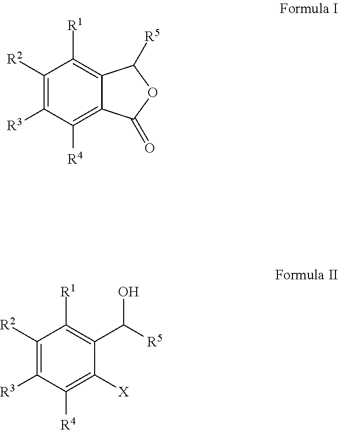

where, R¹ to R⁵ and X are described herein below.

Rosenmund-von Braun Reaction, a known organic reaction, for the preparation of nitrile from aryl halide is extended in the instant invention to prepare 3-substituted phthalides through intramolecular lactonization of hydroxy substituted halobenzene. Accordingly, in the present invention, oxidative cyclisation is effected with the use of CuCN which is cheap, easy to perform at higher scales, shows remarkably broad substrate scope and good functional group tolerance and not much effluent is generated.

The 3-substituted phthalides of formula I is represented as follows:

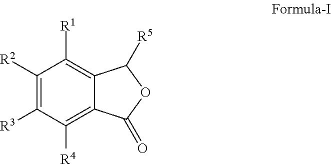

where R¹, R², R³, R⁴ are selected independently from the group consisting of hydrogen, halogen, hydroxy, cyano, carboxyl, amino or substituted amino, alkyl, alkenyl, alkynyl, alkoxy, alkenyloxy, alkynyloxy, alkylthio, alkenylthio, alkynylthio, aryl, aralkyl, aralkenyl, aralkynyl, aryloxy, aralkoxy, acyl, acylalkyl, acyloxy, acyloxyalkyl, hydroxyalkyl, cycloalkyl, alkylthio, alkylsulfonyl, alkylsulfinyl, heteroaryl, which may optionally be substituted with halo, hydroxy, alkyl, alkoxy, nitro, amino, cyano, carboxy;

R⁵ is selected independently from the group consisting of alkyl, alkenyl, alkynyl, aryl, which may optionally be substituted with halo, hydroxy, alkyl, alkoxy, nitro, amino, cyano, carboxy; acyloxyalkyl, acyloxyaryl,

with the proviso, that when R² and R³ together represent —O—CH₂—O—, R¹ and R⁴ are hydrogen, R⁵ is selected independently from alkyl, alkenyl, alkynyl, aryl, which may optionally be substituted with halo, hydroxy, alkyl, alkoxy, nitro, amino, cyano, carboxy; acyloxyalkyl or acyloxyaryl;

with the proviso that when R¹, R², R³ and R⁴ together represent the group selected from (E)-ethyl 3-(1-cyanonaphthalen-2-yl)acrylate; 3-(1-hydroxybut-3-enyl)pyridine-2-carbonitrile; 1-(3-bromofuran-2-yl)but-3-en-1-ol; R⁵ is selected independently from alkyl, alkenyl, alkynyl, aryl, which may optionally be substituted with halo, hydroxy, alkyl, alkoxy, nitro, amino, cyano, carboxy; acyloxyalkyl or acyloxyaryl,

with the proviso when R¹ to R⁴ are similar or independently selected from the group consisting of hydrogen, then R⁵ is particularly selected from the group consisting of (C¹-C¹⁶) alkyl, (C²-C⁸) alkynyl, carboxylate, vinyl, aryl, alkylaryl, optionally be substituted with hydrogen, halo, hydroxy, alkyl, alkoxy, nitro, amino, cyano and like thereof.

Present invention provides a one pot synthesis of various 3-substituted phthalides of formula I and their structural analogues which include reacting a compound of formula II with CuCN in polar aprotic solvent and refluxing the mixture at a temperature in the range of 145-155° C. for 10-12 hours. The compound of formula II is given below;

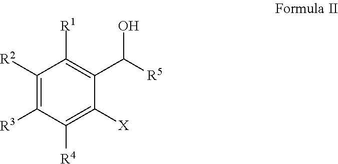

where R¹, R², R³, R⁴ are selected independently from hydrogen, halogen, hydroxy, cyano, carboxyl, amino or substituted amino, alkyl, alkenyl, alkynyl, alkoxy, alkenyloxy, alkynyloxy, alkylthio, alkenylthio, alkynylthio, aryl, aralkyl, aralkenyl, aralkynyl, aryloxy, aralkoxy, acyl, acylalkyl, acyloxy, acyloxyalkyl, hydroxyalkyl, cycloalkyl, alkylthio, alkylsulfonyl, alkylsulfinyl, which may optionally be substituted with halo, hydroxy, alkyl, alkoxy, nitro, amino, cyano, carboxy;

R⁵ is selected independently from alkyl, allyl, vinyl, alkynyl, aryl, alkenyl, which may optionally be substituted with halo, hydroxy, alkyl, alkoxy, nitro, amino, cyano, carboxy; acyloxyalkyl, acyloxyaryl,

X represents a halo group (i.e. Cl, Br or I) preferably Br;

with the proviso, that when R² and R³ together represent —O—CH₂—O—, R¹ and R⁴ are hydrogen, R⁵ is selected independently from alkyl, allyl, vinyl, alkynyl, aryl, alkenyl, which may optionally be substituted with halo, hydroxy, alkyl, alkoxy, nitro, amino, cyano, carboxy; acyloxyalkyl or acyloxyaryl.

with the proviso that when R¹, R², R³ and R⁴ together represent the group selected from (E)-ethyl 3-(1-cyanonaphthalen-2-yl)acrylate; 3-(1-hydroxybut-3-enyl)pyridine-2-carbonitrile; 1-(3-bromofuran-2-yl)but-3-en-1-ol; R⁵ is selected independently from alkyl, allyl, vinyl, alkynyl, aryl, alkenyl, which may optionally be substituted with halo, hydroxy, alkyl, alkoxy, nitro, amino, cyano, carboxy; acyloxyalkyl or acyloxyaryl.

with the proviso when R¹ to R⁴ are similar or independently selected from the group consisting of hydrogen, then R⁵ is particularly selected from the group consisting of (C¹-C¹⁶) alkyl, (C²-C⁸) alkynyl, carboxylate, vinyl, aryl, alkylaryl, optionally be substituted with hydrogen, halo, hydroxy, alkyl, alkoxy, nitro, amino, cyano and like thereof. The process is depicted in Scheme 1.

The process steps involve tandem reaction sequence where in the first step halo is substituted with CN followed by intramolecular lactonization to access 3-substituted phthalides with high yields. The halo group is preferably bromo. The polar aprotic solvent is selected from DMF, DCM, acetone, THF, acetonitrile etc.

In one aspect, the invention provides preparation of halo allyl alcohols (formula-II) as per Scheme 2. In a typical procedure, a pre-cooled mixture of 2-halo aldehydes, Zn dust and allyl bromide in CH₃CN was added to saturated solution of NH₄Cl under stirring. The mixture was stirred at ambient temperature until the aldehyde was totally consumed (monitored by TLC). The mixture was filtered and the precipitate was washed thoroughly with EtOAc. The organic layer is then washed with brine and dried over anhyd. Na₂SO₄. Removal of solvent under reduced pressure gave crude product which on chromatographic separation with petroleum ether/EtOAc yield corresponding halo allyl alcohols in pure form.

In another aspect, the halo allyl alcohol thus obtained was taken in dry DMF and CuCN was added to it and the entire solution was refluxed under N₂ for 12 h (monitored by TLC). The reaction mixture was then cooled to room temperature 25 to 40° C., and diluted with water and EtOAc. The organic layer was separated and the aqueous layer was extracted with EtOAc. The combined organic extracts were washed with brine and dried over anhyd. Na₂SO₄ and concentrated under reduced pressure to give the crude products, which were purified by column chromatography [silica gel (230-400 mesh) and petroleum ether:EtOAc as an eluent] to give 3-substituted phthalide derivatives.

Thus, the invention provides a process for the synthesis of 3-substituted phthalides I (1-13) from the corresponding o-bromobenzylalcohol derivatives II (1′-13′). For example, 1-(2-bromo-3,5-dimethoxyphenyl)but-3-en-1-ol, readily derived from 2-bromo-3,5-dimethoxybenzaldehyde via Barbier allylation, was subjected to Rosenmund-von Braun reaction in the presence of CuCN (3 equiv.) in DMF under reflux condition, the corresponding phthalide was formed in 86% yield (Scheme 3).

Encouraged by the above result, the inventors have established the scope of the reaction by applying the same to various substrates to obtain corresponding phthalides. Accordingly, the invention provides various substituted phthalides from the corresponding bromo alcohols. The process includes subjecting o-bromobenzyl alcohols (Formula II) 1′-13′ to Rosenmund-von Braun reaction in the presence of CuCN (3 equiv) in DMF under reflux condition. 2-Bromobenzyl alcohols (Formula II) 1′-13′ were prepared in one step, starting from the corresponding o-bromoaldehydes via Barbier allylation or Grignard reaction using allylbromide or alkyl halides in 79-88% yield. When subjected to CuCN-mediated “one-pot” cyclization with CuCN (3 equiv) in DMF at 150° C., o-bromobenzyl alcohols (Formula II) 1′-13′ gave the corresponding phthalide derivatives (Formula I) 1-13 in 82-88% yields, the results of which are presented in Table 1. As can be seen, in every case, the reaction proceeded smoothly within 10-12 h giving the desired phthalides (Formula I) 1-13 in excellent yields. For instance, substrates having halogen (Entry 7), highly electron-rich groups (Entry 4) and different alkyl groups at R⁴ (entries 9-13) underwent this cyclization smoothly affording the corresponding phthalides (Formula I) 1-13 in excellent yields.

TABLE 1
CuCN-Mediated One-pot Synthesis of 3-Substituted Phthalides

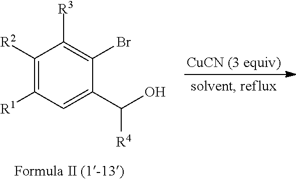

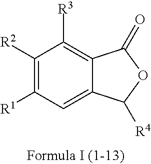

| Entry | R¹ | R² | R³ | R⁴ | Yield (%)^{a} |
|---|---|---|---|---|---|
| 1 | H | H | H | H | 88 |
| 2 | OMe | H | H | H | 86 |
| 3 | OMe | H | OMe | H | 86 |
| 4 | OMe | OMe | OMe | H | 85 |
| 5 | OTs | OMe | H | H | 88 |
| 6 | OBn | OMe | H | H | 82 |
| 7 | F | H | H | H | 88 |
| 8 | —O—CH₂—O— | | H | H | 84 |
| 9 | H | H | H | Me | 88 |
| 10 | H | H | H | n-C₂H₅ | 88 |
| 11 | H | H | H | n-C₃H₇ | 86 |
| 12 | H | H | H | n-C₄H₉ | 85 |
| 13 | H | H | H | n-C₇H₁₅ | 86 |

^{a}Isolated yield after column chromatographic purification. Various 3-substituted phthalides obtained by the process of the current invention is given below in Table 2. (wherein R¹, R², R³ and R⁴ are hydrogen, R⁵ is selected from the group given in Table 2)

TABLE 2

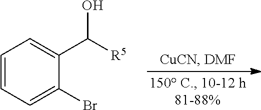

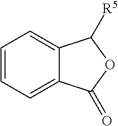

| Entry | R⁵ | Yield (%)^{a} |
|---|---|---|
| 14 | CH₃ | 88 |
| 15 | C₂H₅ | 85 |
| 16 | C₃H₇ | 87 |
| 17 | C₄H₉ | 87 |
| 18 | C₅H₁₁ | 88 |
| 19 | C₆H₁₃ | 88 |
| 20 | C₇H₁₅ | 87 |
| 21 | C₈H₁₇ | 87 |
| 22 | C₉H₁₉ | 85 |
| 23 | C₁₀H₂₁ | 86 |
| 24 | C₁₁H₂₃ | 88 |
| 25 | C₁₂H₂₅ | 88 |
| 26 | C₁₄H₂₉ | 86 |
| 27 | C₁₅H₃₁ | 86 |
| 28 | vinyl | 85 |
| 29 | propargyl | 88 |
| 30 | Ph | 88 |
| 31 | CH₃C₆H₄ | 86 |
| 32 | ethylpropiolate | 88 |
| 33 | CH₂CO₂Et | 86 |

^{a}Isolated yield after column chromatographic purification.

The invention provides conversion of compound of formula II, where R⁵ is propylene to corresponding 3-allyl-phthalide Formula-I and yields thereof as given below in Table 3.

TABLE 3

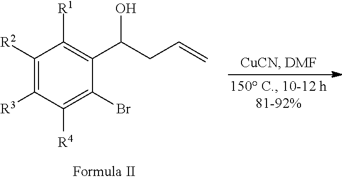

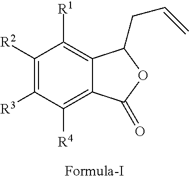

| S. No | R¹ | R² | R³ | R⁴ | Yield (%)^{a} |
|---|---|---|---|---|---|
| 34 | H | H | H | H | 91 |
| 35 | H | OMe | H | H | 89 |
| 36 | H | OMe | OMe | H | 89 |
| 37 | H | H | OMe | OMe | 92 |
| 38 | H | OMe | OMe | OMe | 88 |
| 39 | H | OMe | OMe | OMe | 88 |
| 40 | H | OTs | OMe | H | 86 |
| 41 | H | OBn | OMe | H | 88 |
| 42 | H | H | H | F | 86 |
| 43 | H | NO₂ | H | H | 82 |
| 44 | H | CN | H | H | 81 |
| 45 | OMe | OMe | H | H | 85 |
| 46 | H | Me | Me | H | 86 |
| 47 | H | Me | H | H | 88 |
| 48 | H | Cl | H | H | 81 |
| 49 | H | H | H | OMe | 88 |

| | | | | |
|---|---|---|---|---|
| 50 | H | —O—CH₂—O— | H | 92 |

| | | |
|---|---|---|
| 51 | (E)-ethyl 3-(1-cyanonaphthalen-2-yl)acrylate | 90 |
| 52 | 3-(1-hydroxybut-3-enyl)pyridine-2-carbonitrile | 89 |
| 53 | 1-(3-bromofuran-2-yl)but-3-en-1-ol | 86 |

^{a}Isolated yield after column chromatographic purification.

3-substituted phthalides of formula I according to the invention encompasses for example, 3-Allylisobenzofuran-1-one; 3-Allyl-5-methoxyisobenzofuran-1-one; 3-Allyl-5,7-dimethoxyisobenzofuran-1-one; 3-Allyl-4,5,6-trimethoxyisobenzofuran-1-one; 1-Allyl-1,3-dihydro-5-methoxy-3-oxoisobenzofuran-6-yl4-methylbenzenesulfonate; 3-Allyl-6-methoxy-5-phenoxyisobenzofuran-1-one; 3-Allyl-5-fluoroisobenzofuran-1(3H)-one 7-Allylisobenzofuro[5,6-d][1,3]dioxol-5(7H)-one; 3-Methyl-3H-isobenzofuran-1-one; 3-Ethyl-3H-isobenzofuran-1-one; 3-Propyl-3H-isobenzofuran-1-one; 3-Butylisobenzofuran-1(3H)-one; 3-Heptylisobenzofuran-1(3H)-one etc.

The compound of formula II according the invention, for example selected from the group consisting of 1-(2-Bromophenyl)but-3-en-1-ol; 1-(2-Bromo-5-methoxyphenyl)but-3-en-1-ol; 1-(2-Bromo-3,5-dimethoxyphenyl)but-3-en-1-ol; 1-(2-Bromo-3,4,5-trimethoxyphenyl)but-3-en-1-ol; 4-Bromo-5-(1-hydroxybut-3-enyl)-2-methoxyphenyl-4-methylbenzenesulfonate; 1-(2-Bromo-4-methoxy-5-phenoxyphenyl)but-3-en-1-ol; 1-(2-Bromo-5-fluorophenyl)but-3-en-1-ol; 1-(6-Bromobenzo[d][1,3]dioxol-5-yl)but-3-en-1-ol; 1-(2-Bromophenyl)ethanol; 1-(2-Bromophenyl)-1-propanol; 1-(2-Bromophenyl)butan-1-ol; 1-(2-Bromophenyl)pentan-1-ol; 1-(2-Bromophenyl)octan-1-ol etc.

The invention provides a study on effectiveness of the halo group converting into nitrile and subsequent intramolecular lactonization. Accordingly, the preferable halo group is bromine which gives high yields as depicted in Table 4.

TABLE 4

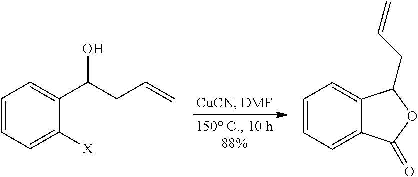

| Entry | X | Time (h) | Yield (%) |
|---|---|---|---|
| 1 | I | 8 h | 82 |
| 2 | Br | 10 h | 91 |
| 3 | CI | 16 h | 75 |

The synthetic potential of this protocol is illustrated with the facile synthesis of natural products like isochracinic acid, paecilocin A and herbaric acid is given in scheme 4, 5 and 6 respectively.

The compounds 3-substituted phthalides prepared by novel, cheap, one pot process of the current invention may be used as antimicrobial against human pathogenic bacteria and fungi. The compounds of the current invention may be formulated into pharmaceutical compositions along with pharmaceutically acceptable excipients and can be delivered to the subject in various forms and in varied dosages by a process known in the art.

Thus the invention provides a novel one-pot tandem route for the synthesis of a wide variety of 3-substituted phthalides and their structural analogues via Rosenmund-von Braun reaction. This reaction is highly practical in the sense that the products were obtained in excellent yields. It also shows broad substrate scope and good functional group tolerance. Therefore, the intramolecular cyclization strategy of the instant invention should find wide applications in the total synthesis of bioactive phthalide frameworks.

### EXAMPLES

Following examples are given by way of illustration therefore should not be construed to limit the scope of the invention.

### General Experimental Procedure for the Preparation of Halo Allyl Alcohols (Formula-II) (1′-13′)

To a pre-cooled (0° C.), well stirred mixture of 2-bromo aldehydes (1 mmol), Zn dust (2 mmol) and allyl bromide (1.8 mmol) in 10 mL of CH₃CN was added a saturated solution of NH₄Cl (1 mL). The mixture was stirred for 10 h at ambient temperature until the aldehyde was totally consumed (monitored by TLC). The mixture was filtered and the precipitate was washed thoroughly with EtOAc (3×10 mL). The organic layer is then washed with brine and dried over anhyd. Na₂SO₄. Removal of solvent under reduced pressure gave crude product which on chromatographic separation with petroleum ether/EtOAc (7:3 v/v) gave halo allyl alcohols (II) (1′-13′) in pure form.

### 1-(2-Bromophenyl)but-3-en-1-ol (1′)

Yield: 88%, colorless oil; IR (CHCl₃, cm⁻¹): uₘₐₓ 792, 865, 985, 1015, 1134, 1323, 1386, 1432, 1476, 1565, 2934, 3425; ¹H-NMR (200 MHz, CDCl₃): δ 2.14 (d, J=4.1 Hz, 1H), 2.25-2.41 (m, 1H), 2.63-2.70 (m, 1H), 5.05-5.14 (m, 2H), 5.22-5.25 (m, 1H), 5.77-5.98 (m, 1H), 7.07-7.16 (m, 1H), 7.28-7.36 (m, 1H), 7.48-7.57 (m, 2H); ¹³C-NMR (50 MHz, CDCl₃): δ 42.0, 71.7, 118.5, 121.7, 127.3, 127.6, 128.7, 132.5, 134.2, 142.7; Analysis: C₁₀H₁₁BrO₁ requires C, 52.89; H, 4.88. Found: C, 52.76; H, 4.72%.

### 1-(2-Bromo-5-methoxyphenyl)but-3-en-1-ol (2′)

Yield: 80%, colorless oil; IR (CHCl₃, cm⁻¹): uₘₐₓ 680, 858, 976, 1025, 1148, 1350, 1386, 1472, 1488, 1575, 2928, 3414; ¹H-NMR (200 MHz, CDCl₃): δ 2.37-2.39 (m, 2H), 2.62-2.68 (m, 1H), 3.83 (s, 3H), 5.04-5.08 (m, 1H), 5.19-5.25 (m, 2H), 5.84-5.97 (m, 1H), 7.71 (dd, J=3.1, 8.9 Hz, 1H), 7.14 (d, J=3.0 Hz, 1H). 7.27 (d, J=8.1 Hz, 1H); ¹³C-NMR (50 MHz, CDCl₃): δ 42.0, 55.5, 71.9, 112.0, 114.9, 118.5, 133.2, 134.3, 143.9, 145.1, 159.2; Analysis: C₁H₁₃BrO₂ requires C, 51.38; H, 5.10. Found: C, 51.29; H, 5.01%.

### 1-(2-Bromo-3,5-dimethoxyphenyl)but-3-en-1-ol (3′)

Yield: 82%, yellow oil; IR (CHCl₃, cm⁻¹): uₘₐₓ 723, 798, 846, 975 1056, 1345, 1392, 1476, 1492, 1568, 2945, 3014, 3398; ¹H-NMR (200 MHz, CDCl₃): δ 2.14 (br s, 1H), 2.22-2.37 (m, 1H), 2.57-2.70 (m, 1H), 3.82 (s, 3H), 3.87 (s, 3H), 5.09-5.24 (m, 3H), 5.22 5.79-6.0 (m, 1H), 6.39 (d, J=2.9 Hz, 1H), 6.70 (d, J=2.9 Hz, 1H); ¹³C-NMR (50 MHz, CDCl₃): δ 41.7, 55.3, 56.0, 71.9, 102.8, 108.1, 134.4, 144.9, 156.0, 159.7; Analysis: C₁₂H₁₅BrO₃ requires C, 50.19; H, 5.27. Found: C, 50.12; H, 5.20%.

### 1-(2-Bromo-3,4,5-trimethoxyphenyl)but-3-en-1-ol (4′)

Yield: 79%, gum; IR (CHCl₃, cm⁻¹): uₘₐₓ 1009, 1105, 1162, 1195, 1324, 1394, 1426, 1481, 1569, 2938, 3435; ¹H-NMR (200 MHz, CDCl₃): δ 2.17 (d, J=4.1 Hz, 1H), 2.24-2.35 (m, 1H), 2.56-2.68 (m, 1H), 3.87 (s, 3H), 3.89 (s, 6H), 5.02-5.10 (m, 1H), 5.14-5.18 (s 1H), 5.21-5.27 (m, 1H), 5.79-6.00 (m, 1H), 6.95 (s, 1H); ¹³C-NMR (50 MHz, CDCl₃): δ 42.2, 56.1, 61.0, 61.1, 71.8, 105.8, 107.8, 118.9, 134.5, 138.5, 142.1, 150.4, 153.0; Analysis: C₁₂H₁₅BrO₄ requires C, 59.23; H, 5.40. Found: C, 59.13; H, 5.29%.

### 4-Bromo-5-(1-hydroxybut-3-enyl)-2-methoxyphenyl-4-methylbenzenesulfonate (5′)

Yield: 84%, yellow oil; IR (CHCl₃, cm⁻¹): uₘₐₓ 790, 826, 888, 986, 1185, 1278, 1384, 1436, 1545, 2927, 3419; ¹H-NMR (200 MHz, CDCl₃): δ 2.16-2.30 (m, 2H), 2.46 (s, 3H), 2.57-2.68 (m, 1H), 3.69 (s, 3H), 4.95-5.02 (m, 1H), 5.13-5.17 (m, 1H), 5.22-5.24 (m, 1H), 5.76-5.97 (m, 1H), 7.09 (s, 1H), 7.29-7.33 (m, 3H), 7.77 (d, J=8.2 Hz, 2H); Analysis: C₁₈H₁₉BrO₅S requires C, 50.59; H, 4.48; S, 7.50. Found: C, 50.43; H, 4.40; S, 7.42%.

### 1-(2-Bromo-4-methoxy-5-phenoxyphenyl)but-3-en-1-ol (6′)

Yield: 86%, yellow oil; IR (CHCl₃, cm⁻¹): uₘₐₓ 694, 756, 852, 894, 974, 1085, 1124, 1267, 1358, 1457, 1542, 2934, 3032, 3424; ¹H-NMR (200 MHz, CDCl₃): δ 2.09 (brs, 1H), 2.22-2.38 (m, 1H), 2.52-2.68 (m, 1H), 3.88 (s, 3H), 4.96-5.02 (m, 1H), 5.02 (s, 2H), 5.12-5.14 (m, 1H), 5.18-5.23 (m, 1H), 5.77-5.98 (m, 1H), 7.00 (s, 1H), 7.08 (s, 1H), 7.34-7.45 (m, 5H); ¹³C-NMR (50 MHz, CDCl₃): δ 42.4, 56.2, 71.2.9, 71.7, 110.3, 111.2, 118.5, 127.4, 128.0, 128.6, 134.4, 135.5, 136.4, 147.8, 149.4; Analysis: C₁₇H₁₇BrO₃ requires C, 58.47; H, 4.91. Found: C, 58.36; H, 4.82%.

### 1-(2-Bromo-5-fluorophenyl)but-3-en-1-ol (7′)

Yield: 82%, gum; IR (CHCl₃, cm⁻¹): uₘₐₓ 774, 826, 878, 989, 1167, 1265, 1376, 1435, 1564, 2985, 3420; ¹H-NMR (200 MHz, CDCl₃): δ 2.20 (br s, 1H), 2.25-2.36 (m, 1H), 2.58-2.71 (m, 1H), 4.99-5.05 (m, 1H), 5.16-5.18 (m, 1H), 5.23-5.27 (m, 1H), 5.77-5.97 (m, 1H), 6.81-6.91 (m, 1H), 7.30 (dd, J=4.1, 4.3 Hz, 1H), 7.46 (dd, J=4, 4.4 Hz, 1H); ¹³C-NMR (50 MHz, CDCl₃): δ 41.9, 71.6, 114.6 (d, J=26 Hz), 115.3, 115.8 (d, J=26 Hz), 119.1, 133.7, 133.8, 145.9, 162.8 (d, J=250 Hz); Analysis: C₁₀H₁₀BrFO requires C, 49.01; H, 4.11. Found: C, 48.96; H, 4.01%.

### 1-(6-Bromobenzo[d][1,3]dioxol-5-yl)but-3-en-1-ol (8′)

Yield: 85%, gum; IR (CHCl₃, cm⁻¹): uₘₐₓ 678, 760, 874, 899, 965, 1084, 1167, 1278, 1339, 1465, 1564, 2928, 3016, 3418; ¹H-NMR (200 MHz, CDCl₃): δ 2.22-2.38 (m, 1H), 2.49-2.61 (m, 1H), 5.01-5.06 (m, 1H), 5.12-5.15 (m, 1H), 5.75-5.87 (m, 1H), 5.96 (s, 2H), 6.95 (s, 1H), 7.04 (s, 1H); ¹³C-NMR (50 MHz, CDCl₃): δ 42.2, 71.7, 101.6, 107.2, 111.8, 118.5, 134.2, 136.2, 147.5; Analysis: C₁₁H₁₁BrO₃ requires C, 48.73; H, 4.09. Found: C, 48.62; H, 4.01%.

### 1-(2-Bromophenyl)ethanol (9′)

Yield: 82%, yellow oil; IR (CHCl₃, cm⁻¹): uₘₐₓ 775, 798, 874, 974, 1072, 1189, 1458, 2916, 3025, 3424; ¹H-NMR (200 MHz, CDCl₃): δ 1.40 (d, J=6.79 Hz, 3H), 3.19 (br s, 1H), 5.16 (q, J=6.04, 6.79 Hz, 1H), 7.04-7.09 (m, 1H), 7.25-7.30 (m, 1H), 7.44-7.54 (m, 2H); ¹³C-NMR (50 MHz, CDCl₃): δ 23.5, 68.9, 121.5, 126.6, 127.7, 128.5, 132.4, 144.5; Analysis: C₈H₉BrO requires C, 47.79; H, 4.51. Found: C, 47.69; H, 4.43%.

### 1-(2-Bromophenyl)-1-propanol (10′)

Yield: 84%, colorless oil; IR (CHCl₃, cm⁻¹): uₘₐₓ 741, 894, 1020, 1466, 2967, 3385; ¹H-NMR (200 MHz, CDCl₃): δ 1.0 (t, J=7.5 Hz, 3H), 1.94-1.59 (m, 2H), 2.09 (br s, 1H), 7.35-7.52 (m, 2H), 5.0 (dd, J=4.8, 7.5 Hz, 1H), 7.11 (dt, J=7.5, 1.6 Hz, 1H), 7.33 (t, J=7.5 Hz, 1H) 7.42-7.53 (m, 2H); ¹³C-NMR (50 MHz, CDCl₃): δ 10.1, 30.5, 74.2, 122.1, 127.3, 127.6, 128.7, 132.6, 143.5; Analysis: C₉H₁₁BrO requires C, 50.26; H, 5.15. Found: C, 50.36; H, 5.09%.

### 1-(2-Bromophenyl)butan-1-ol (11′)

Yield: 79%, colorless oil; IR (CHCl₃, cm⁻¹): uₘₐₓ 723, 876, 1054, 1485, 2956, 3012, 3340; ¹H-NMR (200 MHz, CDCl₃): δ 0.9 (t, J=7.6 Hz, 3H), 1.34-1.56 (m, 2H), 1.94-2.0 (m, 2H), 2.07 (br s, 1H), 7.35-7.52 (m, 2H), 5.0 (dd, J=4.8, 7.5 Hz, 1H), 7.12 (dt, J=1.5, 7.3 Hz, 1H), 7.29 (t, J=7.3 Hz, 1H), 7.42-7.53 (m, 2H); ¹³C-NMR (50 MHz, CDCl₃): δ 10.1, 30.5, 74.2, 122.1, 127.3, 127.6, 128.7, 132.6, 143.5; Analysis: C₁₀H₁₃BrO requires C, 52.42; H, 5.72. Found: C, 50.36; H, 5.12%.

### 1-(2-Bromophenyl)pentan-1-ol (12′)

Yield: 79%, light yellow oil; IR (CHCl₃, cm⁻¹): uₘₐₓ 698, 834, 1037, 1123, 1468, 2918, 3018, 3323; ¹H-NMR (200 MHz, CDCl₃): δ 0.94 (t, J=7.5 Hz, 3H), 1.38 (m, 3H), 1.51 (m, 1H), 1.69 (m, 1H), 1.80 (m, 1H), 2.08 (br s, 1H), 5.07 (dd, J=8.0, 4.5 Hz, 1H), 7.12 (td, J=8.0, 1.5 Hz, 1H), 7.34 (td, J=7.5, 1.0 Hz, 1H), 7.52 (dd, J=8.0, 1.0 Hz, 1H), 7.55 (dd, J=7.5, 1.0 Hz, 1H); ¹³C-NMR (50 MHz, CDCl₃): δ 14.2, 22.7, 28.2, 37.6, 73.1, 122.2, 127.5, 128.9, 132.8, 144.1; Analysis: C₁₁H₁₅BrO requires C, 54.34; H, 6.22. Found: C, 54.26; H, 6.16%.

### 1-(2-Bromophenyl)octan-1-ol (13′)

Yield: 84%, yellow oil; IR (CHCl₃, cm⁻¹): uₘₐₓ 686, 785, 876, 1056, 1278, 1443, 2927, 3025, 3345; ¹H-NMR (200 MHz, CDCl₃): δ 0.84-0.90 (m, 3H), 1.24-1.34 (m, 10H), 1.62-1.78 (m, 2H), 1.87-1.92 (m, 1H), 5.01-5.08 (m, 1H), 7.10-7.14 (m, 1H), 7.28-7.35 (m, 1H), 7.47-7.56 (m, 2H); ¹³C-NMR (50 MHz, CDCl₃): δ 14.1, 22.6, 25.7, 29.2, 29.4, 31.8, 37.7, 72.6, 121.8, 127.3, 127.4, 128.3, 132.3, 144.1; Analysis: C₁₄H₂₁BrO requires C, 58.95; H, 7.42. Found: C, 58.88; H, 7.36%.

### General Experimental Procedure for the Preparation of 3-Substituted Phthalides (Formula-I)/(1-13)

Bromo alcohols (II) 1′-13′ (1 mmol) were taken in dry DMF (10 mL) and CuCN (3 mmol) was added to it and the entire solution refluxed under N₂ for 12 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivatives (Formula I) (1-13) in 82-88% yield.

### 3-Allylisobenzofuran-1-one (1)

3-Allylisobenzofuran-1-one (1), (1 mmol) was taken in dry DMF (10 mL) and CuCN (3 mmol) was added to it and the entire solution refluxed under N₂ for 12 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 88%, yellow oil; IR (CHCl₃, cm⁻¹): uₘₐₓ 739, 999, 1060, 1282, 1460, 1597, 1616, 1764, 2982, 3058; ¹H-NMR (200 MHz, CDCl₃): δ 2.60-2.81 (m, 2H), 5.13-5.24 (m, 2H), 5.50 (t, J=7.1 Hz, 1H), 5.65-5.86 (m, 1H), 7.43-7.55 (m, 2H), 7.61-7.69 (m, 1H), 7.87-7.91 (m, 1H); ¹³C-NMR (50 MHz, CDCl₃): δ 38.8, 80.3, 119.8, 122.1, 125.8, 126.4, 129.3, 131.3, 134.1, 149.5, 170.4; ESI-MS: m/z 197.0491 [M+Na]⁺; Analysis: C₁₁H₁₀₀, requires C, 75.84; H, 5.79. Found: C, 75.72; H, 5.68.

### 3-Allyl-5-methoxyisobenzofuran-1-one (2)

3-Allyl-5-methoxyisobenzofuran-1-one (2), (1.2 mmol) were taken in dry DMF (10 mL) and CuCN (3.6 mmol) was added to it and the entire solution refluxed under N, for 12 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 86%, yellow oil; IR (CHCl₃, cm⁻¹): uₘₐₓ 785, 835, 886, 1064, 1282, 1340, 1562, 1645, 1759, 2968, 3032; ¹H-NMR (200 MHz, CDCl₃): δ 2.55-2.80 (m, 2H), 3.90 (s, 3H), 5.12-5.16 (m, 1H), 5.17-5.25 (m, 1H), 5.41 (t, J=5.92 Hz, 1H), 5.67-5.88 (m, 1H), 6.86-6.88 (m, 1H), 6.99-7.04 (m, 1H), 7.77-7.81 (d, J=8.43 Hz, 1H); ¹³C-NMR (50 MHz, CDCl₃): δ 38.8, 55.7, 79.4, 106.1, 116.3, 118.7, 127.2, 131.3, 152.0, 164.5, 169.8; ESI-MS: m/z 227.0614 [M+Na]⁺; Analysis: C₁₂H₁₂O₃ requires C, 70.57; H, 5.92. Found: C, 70.50; H, 5.84%.

### 3-Allyl-5,7-dimethoxyisobenzofuran-1-one (3)

3-Allyl-5,7-dimethoxyisobenzofuran-1-one (3), (1.3 mmol) were taken in dry DMF (10 mL) and CuCN (3.9 mmol) was added to it and the entire solution refluxed under N₂ for 12 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 86%, gum; IR (CHCl₃, cm⁻¹): uₘₐₓ 684, 740, 835, 890, 1027, 1255, 1266, 1335, 1474, 1508, 1752, 2922, 3015; ¹H-NMR (200 MHz, CDCl₃): δ 2.53-2.75 (m, 2H), 3.89 (s, 3H), 3.95 (s, 3H), 5.12-5.17 (m, 1H), 5.16-5.22 (m, 1H), 5.31 (t, J=5.8 Hz, 1H), 5.61-5.85 (m, 1H), 6.39-6.44 (m, 2H); ¹³C-NMR (50 MHz, CDCl₃): δ 38.4, 55.7, 78.6, 98.5, 106.5, 119.0, 131.2, 154.1, 159.3, 166.5, 167.9; Analysis: C₁₃H₁₄O₄ requires C, 66.66; H, 6.02. Found: C, 66.58; H, 5.96%.

### 3-Allyl-4,5,6-trimethoxyisobenzofuran-1-one (4)

3-Allyl-4,5,6-trimethoxyisobenzofuran-1-one (4), (1.0 mmol) were taken in dry DMF (12 mL) and CuCN (3.0 mmol) was added to it and the entire solution refluxed under N, for 12 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 85%, gum; IR (CHCl₃, cm⁻¹): uₘₐₓ 686, 788, 852, 1028, 1278, 1345, 1566, 1634, 1758, 2928, 3025; ¹H-NMR (200 MHz, CDCl₃): δ 2.54-2.69 (m, 2H), 3.86 (s, 3H), 3.93 (s, 3H), 4.13 (s, 3H), 5.13-5.24 (m, 2H), 5.30 (t, J=6.2 Hz, 1H), 5.68-5.89 (m, 1H), 6.60 (s, 1H); ¹³C-NMR (50 MHz, CDCl₃): δ 38.9, 56.3, 61.2, 62.2, 78.7, 99.3, 110.7, 119.4, 131.4, 141.8, 147.1, 152.3, 159.4, 167.5; Analysis: C₁₄H₁₆O₅ requires C, 63.63; H, 6.10. Found: C, 63.52.76; H, 6.01%.

### 1-Allyl-1,3-dihydro-5-methoxy-3-oxoisobenzofuran-6-yl4-methylbenzenesulfonate (5)

1-Allyl-1,3-dihydro-5-methoxy-3-oxoisobenzofuran-6-yl4-methylbenzenesulfonate (5), (1.0 mmol) were taken in dry DMF (12 ml) and CuCN (3.0 mmol) was added to it and the entire solution refluxed under N, for 12 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 78%, gum; IR (CHCl₃, cm⁻¹): uₘₐₓ 764, 835, 873, 925, 1038, 1136, 1265, 1340, 1565, 1628, 1758, 2948, 3016; ¹H-NMR (200 MHz, CDCl₃): δ 2.47 (s, 3H), 2.61-2.72 (m, 2H), 3.78 (s, 3H), 5.15 (s, 1H), 5.21-5.25 (m, 1H), 5.41 (t, J=4.2 Hz, 1H), 5.68-5.87 (m, 1H), 6.88 (s, 1H), 7.34 (d, J=8.2 Hz, 2H), 7.47 (s, 1H), 7.77 (d, J=8.2 Hz, 2H); ¹³C-NMR (50 MHz, CDCl₃): δ 21.6, 38.4, 56.2, 79.4, 105.2, 118.1, 119.7, 120.3, 128.3, 129.6, 131.0, 132.8, 139.6, 145.5, 149.8, 157.2, 168.8; Analysis: C₁₉H₁₈O₆S requires C, 60.95; H, 4.85; S, 8.56. Found: C, 60.83; H, 4.76; S, 8.42%.

### 3-Allyl-6-methoxy-5-phenoxyisobenzofuran-1-one (6)

3-Allyl-6-methoxy-5-phenoxyisobenzofuran-1-one (6), (1.1 mmol) were taken in dry DMF (12 mL) and CuCN (3.3 mmol) was added to it and the entire solution refluxed under N, for 12 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 82%, yellow oil; IR (CHCl₃, cm⁻¹): uₘₐₓ 690, 785, 824, 855, 982, 1023, 1109, 1268, 1348, 1538, 1628, 1756, 2928, 3045; ¹H-NMR (200 MHz, CDCl₃): δ 2.54-2.69 (m, 2H), 3.86 (s, 3H), 3.93 (s, 3H), 4.13 (s, 3H), 5.13-5.24 (m, 2H), 5.30 (t, J=6.2 Hz, 1H), 5.68-5.89 (m, 1H), 6.60 (s, 1H); ¹³C-NMR (50 MHz, CDCl₃): δ 38.9, 56.3, 61.2, 62.2, 78.7, 99.3, 110.7, 119.4, 131.4, 141.8, 147.1, 152.3, 159.4, 167.5; Analysis: C₁₈H₁₆O₄ requires C, 72.96; H, 5.44. Found: C, 72.83; H, 5.36%.

### 3-Allyl-5-fluoroisobenzofuran-1(3H)-one (7)

3-Allyl-5-fluoroisobenzofuran-1(3H)-one (7), (1.0 mmol) were taken in dry DMF (10 mL) and CuCN (3.0 mmol) was added to it and the entire solution refluxed under N₂ for 13 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 88%, yellow oil; IR (CHCl₃, cm⁻¹): uₘₐₓ 665, 774, 836, 992, 1034, 1128, 1269, 1568, 1628, 1762, 2980, 3014; ¹H-NMR (200 MHz, CDCl₃): δ 2.62-2.78 (m, 2H), 5.15 (brs, 1H), 5.21-5.25 (m, 1H), 5.48 (t, J=5.9 Hz, 1H), 5.65-5.86 (m, 1H), 7.12-7.28 (m, 2H), 7.89 (dd, J=4.1, 4.3 Hz, 1H); ¹³C-NMR (50 MHz, CDCl₃): δ38.2, 79.2, 109.3 (d, =25 Hz), 117.2 (d, J=25 Hz), 119.8, 122.2, 127.7 (d, J=11 Hz), 130.6, 151.9, 165.2 (d, J=251 Hz); Analysis: C₁₁H₉FO₂ requires C, 68.74; H, 4.72. Found: C, 68.63; H, 4.66%.

### 7-Allylisobenzofuro[5,6-d][1,3]dioxol-5(7H)-one (8)

7-Allylisobenzofuro[5,6-d][1,3]dioxol-5(7H)-one (8), (1.0 mmol) were taken in dry DMF (10 mL) and CuCN (3.0 mmol) was added to it and the entire solution refluxed under N₂ for 13 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 84%, gum; IR (CHCl₃, cm⁻¹): uₘₐₓ 742, 785, 839, 1056, 1278, 1578, 1629, 1762, 2935, 3035; ¹H-NMR (200 MHz, CDCl₃): δ 2.59-2.68 (m, 2H), 5.11-5.15 (m, 1H), 5.17-5.22 (m, 1H), 5.35 (t, J=6.2 Hz, 1H), 6.11 (s, 2H), 6.79 (s, 1H), 7.18 (s, 1H); ¹³C-NMR (50 MHz, CDCl₃): δ 38.7, 79.3, 102.5, 104.3, 120.0, 131.1, 145.8, 149.2, 153.4, 169.5; Analysis: C₁₂H₁₀O₄ requires C, 66.05; H, 4.62. Found: C, 65.92; H, 4.51%.

### 3-Methyl-3H-isobenzofuran-1-one (9)

3-Methyl-3H-isobenzofuran-1-one (9), (1.0 mmol) were taken in dry DMF (10 mL) and CuCN (3.0 mmol) was added to it and the entire solution refluxed under N, for 13 h (monitored by TLC). The reaction mixture was then cooled to 25° C., and diluted with water (30 ml) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 88%, colorless oil; IR (CHCl₃, cm⁻¹): uₘₐₓ 739, 755, 967, 1060, 1282, 1460, 1597, 1597, 1615, 1759, 2932, 2982; ¹H-NMR (200 MHz, CDCl₃): δ 1.65 (d, J=6.6 Hz, 3H), 5.58 (q, J=6.6 Hz, 1H), 7.46 (d, J=7.8 Hz, 1H), 7.53 (t, J=7.5 Hz, 1H), 7.66-7.73 (m, 1H), 7.89 (d, J=7.8 Hz, 1H); ¹³C-NMR (50 MHz, CDCl₃): δ 20.3, 77.7, 121.5, 125.6, 125.7, 129.0, 134.0, 151.1, 170.4; ESI-MS: m/z 171.039 [M+Na]⁺; Analysis: C₉H₈O₂ requires C, 72.96; H, 5.44. Found: C, 72.83; H, 5.36%.

### 3-Ethyl-3H-isobenzofuran-1-one (10)

3-Ethyl-3H-isobenzofuran-1-one (10), (1.1 mmol) were taken in dry DMF (10 mL) and CuCN (3.3 mmol) was added to it and the entire solution refluxed under N₂ for 12 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 88%, yellow oil; IR (CHCl₃, cm⁻¹): uₘₐₓ 737, 754, 965, 1062, 1285, 1460, 1761, 2880, 2940, 2970; ¹H-NMR (400 MHz, CDCl₃): δ 1.01 (t, J=7.6 Hz, 3H), 1.78-1.88 (m, 1H), 2.09-2.18 (m, 1H), 5.47 (dd, J=5.1, 6.6 Hz, 1H), 7.45 (d, J=8.4 Hz, 1H), 7.54 (t, J=7.4 Hz, 1H), 7.69 (t, J=7.8 Hz, 1H), 7.90 (d, J=7.2 Hz, 1H); ¹³C-NMR (100 MHz, CDCl₃): δ 8.7, 27.5, 82.2, 121.7, 125.4, 126.1, 128.9, 133.9, 149.6, 170.6; Analysis: C₁₀H₁₀O₂ requires C, 74.06; H, 6.21. Found: C, 73.98; H, 6.16%.

### 3-Propyl-3H-isobenzofuran-1-one (11)

3-Propyl-3H-isobenzofuran-1-one (11), (1.0 mmol) were taken in dry DMF (10 mL) and CuCN (3.0 mmol) was added to it and the entire solution refluxed under N₂ for 12 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 86%, yellow oil; IR (CHCl₃, cm⁻¹): uₘₐₓ 678, 764, 828, 988, 1078, 1125, 1275, 1562, 1628, 1765, 2935, 3060; ¹H-NMR (200 MHz, CDCl₃): δ 0.9 (t, J=7.6 Hz, 3H), 1.56-1.35 (m, 2H), 1.77-1.68 (m, 1H), 2.01-1.95 (m, 1H), 5.46 (dd, J=4.0, 8.0 Hz, 1H), 7.41 (d, J=7.2 Hz, 1H), 7.50 (t, J=7.2 Hz, 1H), 7.65 (t, J=7.6 Hz, 1H), 7.87 (d, J=7.6 Hz, 1H); ¹³C-NMR (50 MHz, CDCl₃): δ 13.9, 18.4, 34.0, 81.4, 121.9, 125.9, 126.3, 129.2, 133.1, 150.3, 170.8; Analysis: C₁₁H₁₂O₂ requires C, 74.98; H, 6.86. Found: C, 74.89; H, 6.75%.

### 3-Butylisobenzofuran-1(3H)-one (12)

3-Butylisobenzofuran-1(3H)-one (12), (1.01 mmol) were taken in dry DMF (10 mL) and CuCN (3.04 mmol) was added to it and the entire solution refluxed under N₂ for 12 h (monitored by TLC). The reaction mixture was then cooled to 25° C., and diluted with water (30 mL) and EtOAc (25 mL). The organic layer was separated and the aqueous layer was extracted with EtOAc (3×25 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 92%, yellow oil; IR (CHCl₃, cm⁻¹): uₘₐₓ 679, 753, 888, 935, 1068, 1145, 1276, 1320, 1545, 1615, 1773, 2928, 3033; ¹H-NMR (200 MHz, CDCl₃): δ 0.87-0.94 (m, 3H), 1.26-1.50 (m, 4H), 1.66-1.84 (m, 1H), 1.96-2.12 (m, 1H), 5.42-5.48 (dd, J=4, 8 Hz, 1H), 7.39-7.50 (m, 2H), 7.61-7.69 (m, 1H), 7.88 (d, J=7.4 Hz, 1H); ¹³C-NMR (50 MHz, CDCl₃): δ 13.5, 22.1, 26.5, 34.1, 80.9, 121.1, 125.8, 128.6, 133.6, 149.7, 170.0; ESI-MS: m/z 213.0813 [M+Na]⁺; Analysis: C₁₂H₁₄O₂ requires C, 75.76; H, 7.42. Found: C, 75.70; H, 7.34%.

### 3-Heptylisobenzofuran-1(3H)-one (13)

3-Heptylisobenzofuran-1(3H)-one (13), (1.01 mmol) were taken in dry DMF (10 mL) and CuCN (3.04 mmol) was added to it and the entire solution refluxed under N, for 12 h (monitored by TLC). The reaction mixture was then cooled to 25° C., and diluted with water (30 mL) and EtOAc (25 mL). The organic layer was separated and the aqueous layer was extracted with EtOAc (3×25 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 86%, yellow oil; IR (CHCl₃, cm⁻¹): uₘₐₓ 694, 767, 828, 1056, 932, 1130, 1275, 1534, 1625, 1768, 2938, 3018; ¹H-NMR (200 MHz, CDCl₃): δ 0.83-0.90 (m, 3H), 1.26-1.38 (m, 10H), 1.73-1.81 (m, 1H), 1.94-2.09 (m, 1H), 5.42-5.48 (m, 1H), 7.39-7.54 (m, 2H), 7.61-7.69 (m, 1H), 7.88 (d, J=7.4 Hz, 1H); ¹³C-NMR (50 MHz, CDCl₃): δ 13.9, 22.4, 24.7, 28.9, 31.5, 34.6, 81.1, 121.6, 125.4, 126.0, 128.8, 133.7, 149.9, 170.2; Analysis: C₁₅H₂₀O₂ requires C, 77.55; H, 8.68. Found: C, 77.48; H, 8.59%.

### General Experimental Procedure for the Preparation of 3-Substituted Phthalides (Formula-I)/(14-53)

Bromo alcohols (II) 1′-13′ (1 mmol) were taken in dry DMF (10 ml) and CuCN (3 mmol) was added to it and the entire solution refluxed under N₂ for 12 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivatives (Formula I) (14-53) in 81-92% yield.

### 3-Methyl-3H-isobenzofuran-1-one (14)

Corresponding bromo allylic alcohol (9), (1.0 mmol) were taken in dry DMF (10 mL) and CuCN (3.0 mmol) was added to it and the entire solution refluxed under N, for 13 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 88%, colorless oil; IR (CHCl₃, cm⁻¹): uₘₐₓ 739, 755, 967, 1060, 1282, 1460, 1597, 1597, 1615, 1759, 2932; 2982; ¹H-NMR (200 MHz, CDCl₃): δ 1.65 (d, J=6.6 Hz, 3H), 5.58 (q, J=6.6 Hz, 1H), 7.46 (d, J=7.8 Hz, 1H), 7.53 (t, J=7.5 Hz, 1H), 7.66-7.73 (m, 1H), 7.89 (d, J=7.8 Hz, 1H); ¹³C-NMR (50 MHz, CDCl₃): δ 20.3, 77.7, 121.5, 125.6, 125.7, 129.0, 134.0, 151.1, 170.4; ESI-MS: m/z 171.039 [M+Na]⁺; Analysis: C₉H₈O₂ requires C, 72.96; H, 5.44. Found: C, 72.83; H, 5.36%.

### 3-Ethyl-3H-isobenzofuran-1-one (15)

Corresponding bromo allylic alcohol (10), (1.1 mmol) were taken in dry DMF (10 mL) and CuCN (3.3 mmol) was added to it and the entire solution refluxed under N₂ for 12 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 88%, yellow oil; IR (CHCl₃, cm⁻¹): uₘₐₓ 737, 754, 965, 1062, 1285, 1460, 1761, 2880, 2940, 2970; ¹H-NMR (400 MHz, CDCl₃): δ1.01 (t, J=7.6 Hz, 3H), 1.78-1.88 (m, 1H), 2.09-2.18 (m, 1H), 5.47 (dd, J=5.1, 6.6 Hz, 1H), 7.45 (d, J=8.4 Hz, 1H), 7.54 (t, J=7.4 Hz, 1H), 7.69 (t, J=7.8 Hz, 1H), 7.90 (d, J=7.2 Hz, 1H); ¹³C-NMR (100 MHz, CDCl₃): δ 8.7, 27.5, 82.2, 121.7, 125.4, 126.1, 128.9, 133.9, 149.6, 170.6; Analysis: C₁₀H₁₀O₂ requires C, 74.06; H, 6.21. Found: C, 73.98; H, 6.16%.

### 3-Propyl-3H-isobenzofuran-1-one (16)

Corresponding bromo allylic alcohol (11), (1.0 mmol) were taken in dry DMF (10 mL) and CuCN (3.0 mmol) was added to it and the entire solution refluxed under N₂ for 12 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 86%, yellow oil; IR (CHCl₃, cm⁻¹): uₘₐₓ 678, 764, 828, 988, 1078, 1125, 1275, 1562, 1628, 1765, 2935, 3060; ¹H-NMR (200 MHz, CDCl₃): δ 0.9 (t, J=7.6 Hz, 3H), 1.56-1.35 (m, 2H), 1.77-1.68 (m, 1H), 2.01-1.95 (m, 1H), 5.46 (dd, J=4.0, 8.0 Hz, 1H), 7.41 (d, J=7.2 Hz, 1H), 7.50 (t, J=7.2 Hz, 1H), 7.65 (t, J=7.6 Hz, 1H), 7.87 (d, J=7.6 Hz, 1H); ¹³C-NMR (50 MHz, CDCl₃): δ 13.9, 18.4, 34.0, 81.4, 121.9, 125.9, 126.3, 129.2, 133.1, 150.3, 170.8; Analysis: C₁₁H₁₂O₂ requires C, 74.98; H, 6.86. Found: C, 74.89; H, 6.75%.

### 3-Butylisobenzofuran-1(3H)-one (17)

Corresponding bromo allylic alcohol (12), (1.01 mmol) were taken in dry DMF (10 mL) and CuCN (3.04 mmol) was added to it and the entire solution refluxed under N₂ for 12 h (monitored by TLC). The reaction mixture was then cooled to 25° C., and diluted with water (30 mL) and EtOAc (25 mL). The organic layer was separated and the aqueous layer was extracted with EtOAc (3×25 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 92%, yellow oil; IR (CHCl₃, cm⁻¹): uₘₐₓ 679, 753, 888, 935, 1068, 1145, 1276, 1320, 1545, 1615, 1773, 2928, 3033; ¹H-NMR (200 MHz, CDCl₃): δ 0.87-0.94 (m, 3H), 1.26-1.50 (m, 4H), 1.66-1.84 (m, 1H), 1.96-2.12 (m, 1H), 5.42-5.48 (dd, J=4, 8 Hz, 1H), 7.39-7.50 (m, 2H), 7.61-7.69 (m, 1H), 7.88 (d, J=7.4 Hz, 1H); ¹³C-NMR (50 MHz, CDCl₃): δ 13.5, 22.1, 26.5, 34.1, 80.9, 121.1, 125.8, 128.6, 133.6, 149.7, 170.0; ESI-MS: m/z 213.0813 [M+Na]⁺; Analysis: C₁₂H₁₄O₂ requires C, 75.76; H, 7.42. Found: C, 75.70; H, 7.34%.

### 3-Heptylisobenzofuran-1(3H)-one (20)

Corresponding bromo allylic alcohol (13), (1.01 mmol) were taken in dry DMF (10 mL) and CuCN (3.04 mmol) was added to it and the entire solution refluxed under N₂ for 12 h (monitored by TLC). The reaction mixture was then cooled to 25° C., and diluted with water (30 mL) and EtOAc (25 mL). The organic layer was separated and the aqueous layer was extracted with EtOAc (3×25 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 86%, yellow oil; IR (CHCl₃, cm⁻¹): uₘₐₓ 694, 767, 828, 1056, 932, 1130, 1275, 1534, 1625, 1768, 2938, 3018; ¹H-NMR (200 MHz, CDCl₃): δ 0.83-0.90 (m, 3H), 1.26-1.38 (m, 10H), 1.73-1.81 (m, 1H), 1.94-2.09 (m, 1H), 5.42-5.48 (m, 1H), 7.39-7.54 (m, 2H), 7.61-7.69 (m, 1H), 7.88 (d, J=7.4 Hz, 1H); ¹³C-NMR (50 MHz, CDCl₃): δ 13.9, 22.4, 24.7, 28.9, 31.5, 34.6, 81.1, 121.6, 125.4, 126.0, 128.8, 133.7, 149.9, 170.2; Analysis: C₁₅H₂₀O₂ requires C, 77.55; H, 8.68. Found: C, 77.48; H, 8.59%.

### 3-Allylisobenzofuran-1-one (34)

Corresponding bromo allylic alcohol (1 mmol) was taken in dry DMF (10 mL) and CuCN (3 mmol) was added to it and the entire solution refluxed under N₂ for 12 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 88%, yellow oil; IR (CHCl₃, cm⁻¹): uₘₐₓ 739, 999, 1060, 1282, 1460, 1597, 1616, 1764, 2982, 3058; ¹H-NMR (200 MHz, CDCl₃): δ 2.60-2.81 (m, 2H), 5.13-5.24 (m, 2H), 5.50 (t, J=7.1 Hz, 1H), 5.65-5.86 (m, 1H), 7.43-7.55 (m, 2H), 7.61-7.69 (m, 1H), 7.87-7.91 (m, 1H); ¹³C-NMR (50 MHz, CDCl₃): δ 38.8, 80.3, 119.8, 122.1, 125.8, 126.4, 129.3, 131.3, 134.1, 149.5, 170.4; ESI-MS: m/z 197.0491 [M+Na]*; Analysis: C₁₁H₁₀O₂ requires C, 75.84; H, 5.79. Found: C, 75.72; H, 5.68.

### 3-Allyl-5-methoxyisobenzofuran-1-one (35)

Corresponding bromo allylic alcohol (2), (1.2 mmol) were taken in dry DMF (10 mL) and CuCN (3.6 mmol) was added to it and the entire solution refluxed under N₂ for 12 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 86%, yellow oil; IR (CHCl₃, cm⁻¹): uₘₐₓ 785, 835, 886, 1064, 1282, 1340, 1562, 1645, 1759, 2968, 3032; ¹H-NMR (200 MHz, CDCl₃): δ 2.55-2.80 (m, 2H), 3.90 (s, 3H), 5.12-5.16 (m, 1H), 5.17-5.25 (m, 1H), 5.41 (t, J=5.92 Hz, 1H), 5.67-5.88 (m, 1H), 6.86-6.88 (m, 1H), 6.99-7.04 (m, 1H), 7.77-7.81 (d, J=8.43 Hz, 1H); ¹³C-NMR (50 MHz, CDCl₃): δ 38.8, 55.7, 79.4, 106.1, 116.3, 118.7, 127.2, 131.3, 152.0, 164.5, 169.8; ESI-MS: m/z 227.0614 [M+Na]⁺; Analysis: C₁₂H₁₂O₃ requires C, 70.57; H, 5.92. Found: C, 70.50; H, 5.84%.

### 3-Allyl-5,7-dimethoxyisobenzofuran-1-one (36)

Corresponding bromo allylic alcohol (3), (1.3 mmol) were taken in dry DMF (10 mL) and CuCN (3.9 mmol) was added to it and the entire solution refluxed under N₂ for 12 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 86%, gum; IR (CHCl₃, cm⁻¹): uₘₐₓ 684, 740, 835, 890, 1027, 1255, 1266, 1335, 1474, 1508, 1752, 2922, 3015; ¹H-NMR (200 MHz, CDCl₃): δ 2.53-2.75 (m, 2H), 3.89 (s, 3H), 3.95 (s, 3H), 5.12-5.17 (m, 1H), 5.16-5.22 (m, 1H), 5.31 (t, J=5.8 Hz, 1H), 5.61-5.85 (m, 1H), 6.39-6.44 (m, 2H); ¹³C-NMR (50 MHz, CDCl₃): δ 38.4, 55.7, 78.6, 98.5, 106.5, 119.0, 131.2, 154.1, 159.3, 166.5, 167.9; Analysis: C₁₃H₁₄O₄ requires C, 66.66; H, 6.02. Found: C, 66.58; H, 5.96%.

### 3-Allyl-4,5,6-trimethoxyisobenzofuran-1-one (38)

Corresponding bromo allylic alcohol 4), (1.0 mmol) were taken in dry DMF (12 mL) and CuCN (3.0 mmol) was added to it and the entire solution refluxed under N₂ for 12 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 85%, gum; IR (CHCl₃, cm⁻¹): uₘₐₓ 686, 788, 852, 1028, 1278, 1345, 1566, 1634, 1758, 2928, 3025; ¹H-NMR (200 MHz, CDCl₃): δ 2.54-2.69 (m, 2H), 3.86 (s, 3H), 3.93 (s, 3H), 4.13 (s, 3H), 5.13-5.24 (m, 2H), 5.30 (t, J=6.2 Hz, 1H), 5.68-5.89 (m, 1H), 6.60 (s, 1H); ¹³C-NMR (50 MHz, CDCl₃): δ 38.9, 56.3, 61.2, 62.2, 78.7, 99.3, 110.7, 119.4, 131.4, 141.8, 147.1, 152.3, 159.4, 167.5; Analysis: C₁₄H₁₆O₅ requires C, 63.63; H, 6.10. Found: C, 63.52.76; H, 6.01%.

### 1-Allyl-1,3-dihydro-5-methoxy-3-oxoisobenzofuran-6-yl4-methylbenzenesulfonate (40)

Corresponding bromo allylic alcohol (5), (1.0 mmol) were taken in dry DMF (12 mL) and CuCN (3.0 mmol) was added to it and the entire solution refluxed under N, for 12 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 78%, gum; IR (CHCl₃, cm⁻¹): uₘₐₓ 764, 835, 873, 925, 1038, 1136, 1265, 1340, 1565, 1628, 1758, 2948, 3016; ¹H-NMR (200 MHz, CDCl₃): δ 2.47 (s, 3H), 2.61-2.72 (m, 2H), 3.78 (s, 3H), 5.15 (s, 1H), 5.21-5.25 (m, 1H), 5.41 (t, J=4.2 Hz, 1H), 5.68-5.87 (m, 1H), 6.88 (s, 1H), 7.34 (d, J=8.2 Hz, 2H), 7.47 (s, 1H), 7.77 (d, J=8.2 Hz, 2H); ¹³C-NMR (50 MHz, CDCl₃): δ 21.6, 38.4, 56.2, 79.4, 105.2, 118.1, 119.7, 120.3, 128.3, 129.6, 131.0, 132.8, 139.6, 145.5, 149.8, 157.2, 168.8; Analysis: C₉H₁₈O₆S requires C, 60.95; H, 4.85; S, 8.56. Found: C, 60.83; H, 4.76; S, 8.42%.

### 3-Allyl-6-methoxy-5-phenoxyisobenzofuran-1-one (41)

Corresponding bromo allylic alcohol (6), (1.1 mmol) were taken in dry DMF (12 mL) and CuCN (3.3 mmol) was added to it and the entire solution refluxed under N_{z} for 12 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 82%, yellow oil; IR (CHCl₃, cm⁻¹): uₘₐₓ 690, 785, 824, 855, 982, 1023, 1109, 1268, 1348, 1538, 1628, 1756, 2928, 3045; ¹H-NMR (200 MHz, CDCl₃): δ 2.54-2.69 (m, 2H), 3.86 (s, 3H), 3.93 (s, 3H), 4.13 (s, 3H), 5.13-5.24 (m, 2H), 5.30 (t, J=6.2 Hz, 1H), 5.68-5.89 (m, 1H), 6.60 (s, 1H); ¹³C-NMR (50 MHz, CDCl₃): δ 38.9, 56.3, 61.2, 62.2, 78.7, 99.3, 110.7, 119.4, 131.4, 141.8, 147.1, 152.3, 159.4, 167.5; Analysis: C₁₈H₁₆O₄ requires C, 72.96; H, 5.44. Found: C, 72.83; H, 5.36%.

### 3-Allyl-5-fluoroisobenzofuran-1(3H)-one (42)

Corresponding bromo allylic alcohol (7), (1.0 mmol) were taken in dry DMF (10 mL) and CuCN (3.0 mmol) was added to it and the entire solution refluxed under N₂ for 10-12 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 88%, yellow oil; IR (CHCl₃, cm⁻¹): uₘₐₓ 665, 774, 836, 992, 1034, 1128, 1269, 1568, 1628, 1762, 2980, 3014; ¹H-NMR (200 MHz, CDCl₃): δ 2.62-2.78 (m, 2H), 5.15 (brs, 1H), 5.21-5.25 (m, 1H), 5.48 (t, J=5.9 Hz, 1H), 5.65-5.86 (m, 1H), 7.12-7.28 (m, 2H), 7.89 (dd, J=4.1, 4.3 Hz, 1H); ¹³C-NMR (50 MHz, CDCl₃): δ38.2, 79.2, 109.3 (d, J=25 Hz), 117.2 (d, J=25 Hz), 119.8, 122.2, 127.7 (d, J=11 Hz), 130.6, 151.9, 165.2 (d, J=251 Hz); Analysis: C₁₁H₉FO₂ requires C, 68.74; H, 4.72. Found: C, 68.63; H, 4.66%.

### 7-Allylisobenzofuro[5,6-d][1,3]dioxol-5(7H)-one (50)

Corresponding bromo allylic alcohol (8), (1.0 mmol) were taken in dry DMF (10 mL) and CuCN (3.0 mmol) was added to it and the entire solution refluxed under N₂ for 13 h (monitored by TLC). The reaction mixture was then cooled to 25° C., and diluted with water (30 mL) and EtOAc (25 mL). The organic layer was separated and the aqueous layer was extracted with EtOAc (3×20 mL). The combined organic extracts were washed with brine and dried over anhyd. Na₂SO₄ and concentrated under reduced pressure to give the crude products, which were purified by column chromatography [silica gel (230-400 mesh) and petroleum ether:EtOAc (7:3) as an eluent] to give 3-substituted phthalide derivative.

Yield: 84%, gum; IR (CHCl₃, cm⁻¹): uₘₐₓ 742, 785, 839, 1056, 1278, 1578, 1629, 1762, 2935, 3035; ¹H-NMR (200 MHz, CDCl₃): δ 2.59-2.68 (m, 2H), 5.11-5.15 (m, 1H), 5.17-5.22 (m, 1H), 5.35 (t, J=6.2 Hz, 1H), 6.11 (s, 2H), 6.79 (s, 1H), 7.18 (s, 1H); ¹³C-NMR (50 MHz, CDCl₃): δ 38.7, 79.3, 102.5, 104.3, 120.0, 131.1, 145.8, 149.2, 153.4, 169.5; Analysis: C₁₂H₁₀O₄ requires C, 66.05; H, 4.62. Found: C, 65.92; H, 4.51%.

### Example 3

### Preparation of 3-allylisobenzofuran-1(3H)-one

To a stirred solution of 1-(2-bromophenyl)but-3-en-1-ol (1 mmol) in DMF (10 mL), CuCN (3 mmol) was added and refluxed under N2 atmosphere for 10 h (monitored by TLC). The reaction mixture cooled to room temperature i.e. 25 to 40° C., then diluted with water (10 mL) and EtOAc (15 mL). The organic layer was separated and the aqueous layer was extracted with EtOAc (2×20 mL). The combined organic extracts were washed with brine and dried over anhyd. Na₂SO₄ and concentrated under reduced pressure to give crude products which was purified by column chromatography [silica gel (230-400 mesh) and petroleum ether:EtOAc (70:30) as an eluent] gave 3-allylisobenzofuran-1(3H)-one in 91% yield.

### ADVANTAGES OF THE INVENTION

The development of efficient synthetic procedures for the facile construction of phthalide framework is an important goal in organic synthesis. Inventions have been made in the synthesis of phthalides that exhibited antibacterial and antifungal activity on antimicrobial screening against human pathogenic bacteria and fungi. The reported methods for the synthesis of 3-substituted phthalides employ either lithiation followed by carboxylation or carbamate formation followed by lithiation. This limits the broad substrate scope and higher reaction stereoselectivity. In this context, a more practical and efficient synthesis of functionalized 3-substituted phthalide derivatives is highly desirable using less number of reagents The present inventors have now developed a cheaper and practical protocol for the construction of a wide variety of 3-substituted phthalides and their structural analogues, which have promising pharmacological utility that proceeds with high yields in a single step.

In the present invention, oxidative cyclisation is effected with the use of CuCN which is cheap, easy to perform at higher scales, shows remarkably broad substrate scope and has good functional group tolerance and is not much effluent.

A wide range of natural products with broad, potent, and potentially path-pointing biological activities possess 3-substituted phthalide core. For example, the natural products like isochracinic acid, paecilocin A and herbaric acid which possess 3-substituted phthalide core have antibacterial, antifungal, antibiotic activity. This process provides easy access to these compounds.

1. One pot process for preparation of 3-substituted phthalides of formula I, the said process comprising the steps of reacting halo alcohols of a compound of formula II with CuCN in the ratio ranging between 3.0 to 3.1 in a polar aprotic solvent at a temperature in the range of 145°-155° C. for period in the range of 10-13 hours; 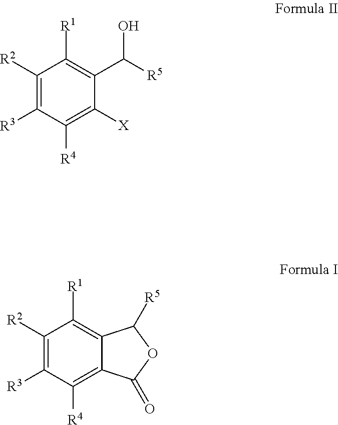
wherein R1, R2, R3, R4 are selected independently from hydrogen, halogen, hydroxy, cyano, carboxyl, amino or substituted amino, alkyl, alkenyl, alkynyl, alkoxy, alkenyloxy, alkynyloxy, alkylthio, alkenylthio, alkynylthio, aryl, aralkyl, aralkenyl, aralkynyl, aryloxy, aralkoxy, acyl, acylalkyl, acyloxy, acyloxyalkyl, hydroxyalkyl, cycloalkyl, alkylthio, alkylsulfonyl, or alkylsulfinyl, which may optionally be substituted with halo, hydroxy, alkyl, alkoxy, nitro, amino, cyano, or carboxy;
R5 is selected independently from alkyl, allyl, vinyl, alkynyl, aryl, alkenyl, which may optionally be substituted with halo, hydroxy, alkyl, alkoxy, nitro, amino, cyano, or carboxy; acyloxyalkyl, or acyloxyaryl; and
X represents a halo group;
with the proviso, that when R2 and R3 together represent —O—CH2-O—, R1 and R4 are hydrogen, R5 is selected independently as described above;
with the proviso that when R1, R2, R3 and R4 together represent the group selected from (E)-ethyl 3-(1-cyanonaphthalen-2-yl)acrylate; 3-(1-hydroxybut-3-enyl)pyridine-2-carbonitrile; and 1-(3-bromofuran-2-yl)but-3-en-1-ol; R5 is selected independently from alkyl, allyl, vinyl, alkynyl, aryl, alkenyl, which may optionally be substituted with halo, hydroxy, alkyl, alkoxy, nitro, amino, cyano, or carboxy; acyloxyalkyl or acyloxyaryl;
with the proviso that when R1 to R4 are the same or independently hydrogen, then R5 is selected from the group consisting of (C1-C16) alkyl, (C2-C8) alkynyl, carboxylate, vinyl, aryl, and alkylaryl, which optionally may be substituted with hydrogen, halo, hydroxy, alkyl, alkoxy, nitro, amino or cyano.

2. The process according to claim 1, wherein the polar aprotic solvent is selected from the group consisting of Dimethylformamide (DMF), Dichloromethane (DCM), acetone, tetrahydrofuran (THF) and acetonitrile.

3. The process according to claim 1, wherein the halo group is selected from the group consisting of chloro, bromo and iodo.

4. The process according to claim 1, further comprising a step of preparing the compound of formula II, wherein the step comprises: subjecting o-bromoaldehydes to Barbier allylation or Grignard reaction using alkyl halides.

5. The process according to claim 1, wherein 3-substituted phthalides of formula I comprises:
i. 3-Allylisobenzofuran-1-one;
ii. 3-Allyl-5-methoxyisobenzofuran-1-one;
iii. 3-Allyl-5,7-dimethoxyisobenzofuran-1-one;
iv. 3-Allyl-4,5,6-trimethoxyisobenzofuran-1-one;
v. 1-Allyl-1,3-dihydro-5-methoxy-3-oxoisobenzofuran-6-yl4-methylbenzenesulfonate;
vi. 3-Allyl-6-methoxy-5-phenoxyisobenzofuran-1-one;
vii. 3-Allyl-5-fluoroisobenzofuran-1 (3H)-one;
viii. 7-Allylisobenzofuro[5,6-d][1,3]dioxol-5(7H)-one;
ix. 3-Methyl-3H-isobenzofuran-1-one; 3-Ethyl-3H-isobenzofuran-1-one;
x. 3-Propyl-3H-isobenzofuran-1-one; 3-Butylisobenzofuran-1 (3H)-one; and
xi. 3-Heptylisobenzofuran-1 (3H)-one.

6. The process as claimed in claim 1, wherein yield of compound of formula I is in the range of 80 to 92%.

7. The process according to claim 1, wherein the compound of formula II is selected from the group consisting of 1-(2-Bromophenyl)but-3-en-1-ol; 1-(2-Bromo-5-methoxyphenyl)but-3-en-1-ol; 1-(2-Bromo-3,5-dimethoxyphenyl)but-3-en-1-ol; 1-(2-Bromo-3,4,5-trimethoxyphenyl)but-3-en-1-ol; 4-Bromo-5-(1-hydroxybut-3-enyl)-2-methoxyphenyl-4-methylbenzenesulfonate; 1-(2-Bromo-4-methoxy-5-phenoxyphenyl)but-3-en-1-ol; 1-(2-Bromo-5-fluorophenyl)but-3-en-1-ol; 1-(6-Bromobenzo[d][1,3]dioxol-5-yl)but-3-en-1-ol; 1-(2-Bromophenyl)ethanol; 1-(2-Bromophenyl)-1-propanol; 1-(2-Bromophenyl)butan-1-ol; 1-(2-Bromophenyl)pentan-1-ol; and 1-(2-Bromophenyl)octan-1-ol.

8. The process according to claim 1, wherein the process comprises: subjecting the bromo alcohols of formula II to Rosenmund-von Braun reaction in the presence of CuCN in DMF under reflux condition.

9. A compound selected from the group consisting of:
a) 3-Allyl-5-methoxyisobenzofuran-1-one;
b) 3-Allyl-4,5,6-trimethoxyisobenzofuran-1-one;
c) 1-Allyl-1,3-dihydro-5-methoxy-3-oxoisobenzofuran-6-yl4-methylbenzenesulfonate;
d) 3-Allyl-6-methoxy-5-phenoxyisobenzofuran-1-one;
e) 3-Allyl-5-fluoroisobenzofuran-1 (3H)-one;
f) 7-Allyl isobenzofuro[5,6-d][1,3]dioxol-5(7H)-one;
g) 3-Ethyl-3H-isobenzofuran-1-one; and
h) 3-Propyl-3H-isobenzofuran-1-one.

10. The process according to claim 3, wherein the halo group is bromo.

11. The process according to claim 4, wherein the o-bromoaldehydes is subjected to Barbier allylation or Grignard reaction using allylbromide.
